(19) United States
(12) Patent Application Publication
(10) Pub. No.: US 2008/0044812 A1
(43) Pub. Date: Feb. 21, 2008

(54) Melting Temperature Dependent Dna Amplification

(76) Inventors: Peter Laurence Molloy, Chatswood (AU); Keith Rand, Frenchs Forest (AU); Susan Joy Clark, Chatswood (AU)

Correspondence Address: SUGHRUE MION, PLLC, 2100 PENNSYLVANIA AVENUE, N.W., SUITE 800, WASHINGTON, DC, 20037, US

(21) Appl. No.: 10/505,773

(22) PCT Filed: Feb. 26, 2003
(86) PCT No.: PCT/AU03/00243

§ 371 Date: Apr. 21, 2005

(30) Foreign Application Priority Data
Feb. 26, 2002 (AU) PS 0769

(51) Int. Cl. C12Q 1/68; C07H 21/04

(52) U.S. Cl. 435/6; 536/25.3

(57) ABSTRACT

A method for the selective amplification of at least one target nucleic acid in a sample comprising a mixture of at least one target nucleic acid and at least one non-target nucleic acid. The method comprises: a nucleic acid denaturation step, wherein the denaturation step is carried out at a temperature at or above the melting temperature of the at least one target nucleic acid but below the melting temperature of the at least one non-target nucleic acid an amplification step using at least one amplification primer.

### FIELD OF THE INVENTION

The present invention relates to a method for nucleic acid amplification. The invention is particularly concerned with a novel selective nucleic acid amplification methods and to the application of those methods.

### BACKGROUND OF THE INVENTION

The polymerase chain reaction (PCR) is based on repeated cycle(s) of denaturation of double stranded DNA, followed by oligonucleotide primer annealing to the DNA template, and primer extension by a DNA polymerase (eg see Mullis el al U.S. Pat. Nos. 4,683,195, 4,683,202 and 4,800,159). The oligonucleotide primers used in PCR are designed to anneal to opposite strands of the DNA, and are positioned so that the DNA polymerase-catalyzed extension product of one primer can serve as a template stand for the other primer. The PCR amplification method results in the exponential increase of discrete DNA the length of which is defined by the 5′ ends of the oligonucleotide primers.

In PCR, reaction conditions are routinely cycled between three temperatures; a high temperature to melt (denature) the double-stranded DNA fragments (usually in the range 90° to 100° C.) followed by a temperature chosen to promote specific annealing of primers to DNA (usually in the range 50° to 70° C.) and finally incubation at an optimal temperature for extension by the DNA polymerase (usually 60° to 72° C.). The choice of primers, annealing temperatures and buffer conditions are used to provide selective amplification of target sequences.

In our cop ending International application entitled “Headloop DNA amplification” filed on 25 Feb. 2003, the entire disclosure of which is incorporated herein by reference, we describe the of method for the selective amplification of a nucleic acid using a primer that includes a region that is an inverted repeat of a sequence in a non-target nucleic acid.

The present inventors have discovered that selective amplification of a nucleic acid can also be achieved by varying the denaturation temperature. The melting temperature of a PCR product depends on its length (increasing length, increasing melting temperature) and its base composition (increasing G+C content, increasing melting temperature). Essentially, the present inventors have realized that amplification of DNA fragments that have a melting temp re higher than that used for denaturation can be suppressed. Whilst differences in melting profiles have been used previously to distinguish and/or identify PCR amplification products, as far as we are aware melting temperature differences have not been used to provide for selective amplification.

### SUMMARY OF THE INVENTION

In a first aspect, the present invention provides a method for the selective amplification of at least one target nucleic acid in a sample comprising the at least one target nucleic acid and at least one non-target nucleic acid, the target nucleic acid having a lower melting point than that of the non-target nucleic acid, the method comprising one or more cycle(s) of a nucleic acid denaturation step followed by an amplification step using at least one amplification primer, wherein the denaturation step is carried out at a temperature at or above the melting temperature of the at least one target nucleic acid but below the melting temperature of the at least one non-target nucleic acid, so as to subs y suppress amplification of the non-target nucleic acid.

The nucleic acid may be DNA.

### DETAILED DESCRIPTION OF THE INVENTION

The method of the present invention may involve the use of a single primer, although it is preferred that the amplification be “exponential” and so utilize a pair of primers, generally referred to as “forward” and “reverse” primers, one of which is complementary to a nucleic acid strand and the other of which is complementary to the complement of that strand.

The method of the present invention may involve the use of a methylate specific primer.

The amplification step of the method may be performed by any suitable amplification technique.

The amplification step may be achieved by a polymerase chain reaction (PCR), a strand displacement reaction (SDA), a nucleic acid sequence-based amplification (NABS), ligation-mediated PCR, and a rolling-circle amplification (RCA).

Preferably, the amplification technique is PCR or the like. The PCR may be any PCR technique, including but not limited to real time PCR.

The selective amplification method of the present invention may be performed on any sample containing target and non-target nucleic acid in which there is a difference in melting points between the target and non-target nucleic acid. This melting point difference may be inherent in the nucleic acids or it may be created or accentuated by modification of one and/or both of the target and non-target nucleic acid(s). This modification may be a chemical modification, for example, by converting one or more bases of the nucleic acids to effect a change in the melting point of the nucleic acid. An example of chemical modification is bisulfate treatment as described in more detail below.

The denaturation temperature used is preferably between the melting temperature of the target and non-target nucleic acids. More preferably, the temperature at which denaturation is carried out is below the melting temperature of the non-target nucleic acid but at or above the melting temperature of the target nucleic acid so as to allow the amplification of the target nucleic acid.

The selective amplification method of the present invention has a wide range of possible applications. For example, by amplifying short DNA fragments, the invention can be applied to the detection of small deletions and base changes and for selectively amplifying different, but related DNA sequences (such as members of multigene families). This could be critical if priming sites are identical for target and non-target. The method of the present invention also has application in diagnostic analysis of mutations and polymorphisms and in analyzing individual members of related genes. The present invention can also be applied for selective amplification of genes from genomes of particular species in mixed DNA samples.

Moreover the present invention can also be used to suppress amplification of spurious PCR products commonly seen in PCR reactions, where those PCR products have a higher melting temperature than the desired product.

Because the denaturation step in the present method can be carried out at lower temperature than in conventional PCR, there is an additional advantage in that the use of lower melting temperatures means that polymerase enzymes will lose activity less rapidly and can potentially be used in lower amounts.

Prior to the amplification step, the method of the invention may include a step of contacting the nucleic acids in the sample with at least one modifying agent so as to change the relative melting temperatures of the at least one target nucleic acid and the at least non-target nucleic acid.

The modification by the modifying agent may increase the difference in melting temperature between the target nucleic acid and the non-target nucleic acid.

Accordingly, in a second aspect the present invention provides a method of the first aspect, wherein the target nucleic acid and/or non-target nucleic acid in the sample has been subjected to a modification step to establish a melting temperature difference or increase the melting temperature difference between the target nucleic acid and the non-target nucleic acid.

Preferably the modification step reduces the melting temperature of a target nucleic acid.

Preferably the modification step changes the relative melting temperatures of the at least one target nucleic acid and the at least one non-target nucleic acid. Where the melting temperatures of the at least one target nucleic acid and the at least one non-target nucleic acid are not substantially different the modification step may increase the difference in melting temperatures. The modification step may modify the at least one target nucleic acid and the at least one non-target nucleic acid to varying degrees.

The modification may be a chemical modification of the nucleic acid. The nucleic acid may comprise methylated and unmethylated cytosines.

Thus, in a third aspect, the present invention provides a method of the second aspect, wherein the nucleic acid in the sample has been contacted with a modifying agent that modifies unmethylated cytosine to produce a converted nucleic acid.

The modifying agent may be a bisuphite.

For example, the method of the present invention has particular application to improving the specificity of amplification of bisulphite-treated DNA By reducing the temperature used to denature DNA fragments in PCR we have been able to eliminate or suppress those unwanted products that have a higher melting temperature than the desired target Such products may be non-converted or partially converted DNA.

It is to be understood that the present invention is not restricted in its application to bisulphite-modified DNA.

A particular, but not exclusive application of the method of the invention is to assay or detect site abnormalities in the nucleic acid sequences, including abnormal under-methylation.

Studies of gene expression have previously suggested a strong correlation between methylation of regulatory regions of genes and many diseases or conditions, including many forms of cancer. Indeed some diseases are characterized by abnormal methylation of cytosine at a site or sites within the glutathione-S-transferase (GSTP1) gene and/or its regulatory flanking sequences, The effects of abnormal methylation of the GSTP1 genes are disclosed in WO 9955905, the entire disclosure of which is herein incorporated by reference.

Methyl insufficiency and/or abnormal DNA methylation has been implicated in development of various human pathologies including cancer. Abnormal methylation in the form of hypomethylation has been linked with diseases and cancers. Examples of cancers in which hypomethylation has been implicated are lung cancers, breast cancer, cervical dysplasia and carcinoma, colorectal cancer, prostate cancer and liver cancer. See for example, Cui et al Cancer Research, Vol 62, p 6442, 2002; Gupta et al, Cancer Research, Vol. 63, p 664 2003; Scelfo et al Oncogen, Vol 21, p 2654.

The method of the present invention may be used as an assay for abnormal methylation, where the abnormal methylation is under-methylation.

Accordingly, in another aspect, the present invention provides an assay for abnormal under-methylation of nucleic acids, wherein said assay comprises the steps of
i) reacting isolated nucleic acid(s) with bisulphite
ii) performing a selective amplification of nucleic acids from (i) wherein the selective amplification comprises one or more cycle(s) of a denaturation step prior to an amplification step, wherein the denaturation is carried out at a temperature at or above the melting temperature of target nucleic acid containing abnormally under-methylated nucleic acids but below the melting temperature of non-target methylated or substantially methylated nucleic acid(s) so as to substantially suppress amplification of the non-target nucleic acid; and
iii) determining the presence of amplified nucleic acid.

The nucleic acid may be DNA.

In another aspect, the present invention provides a diagnostic or prognostic assay for a disease or cancer in a subject, said disease or condition characterized by abnormal under-methylation of nucleic acids, wherein said assay comprises the steps of
i) reacting isolated nucleic acid(s) with bisulphite
ii) performing a selective amplification of nucleic acids from (i) wherein the selective amplification comprises one or more cycle(s) of a denaturation step prior to an amplification step, wherein the denaturation is carried out at a temperature at or above the melting temperature of target nucleic acid containing abnormally under-methylated nucleic acids but below the melting temperature of non-target methylated or substantially methylated nucleic acid(s) so as to substantially suppress amplification of the non-target nucleic acid; and
iii) determining the presence of amplified nucleic acid.

The assay of the latter aspect may used for prognosis or diagnosis of a cancer characterised by undermethylation of nucleic acid. The cancer may be lung cancers, breast cancer, cervical dysplasia and carcinoma, colorectal cancer, prostate cancer and liver cancer.

Terminology

The term “primer” as used in the present application, refers to an oligonucleotide which is capable of acting as a point of initiation of synthesis in the presence of nucleotide and a polymerization agent. The primers are preferably single stranded but may be double stranded. If the primers are double stranded, the strands are separated prior to the amplification reaction. The primers used in the present invention, are selected so that they are sufficiently complementary to the different strands of the sequence to be amplified that the primers are able to hybridize to the strands of the sequence under the amplification reaction conditions. Thus, noncomplementary bases or sequences can be included in the primers provided that the primers are sufficiently complementary to the sequence of interest to hybridize to the sequence.

The oligonucleotide primers can be prepared by methods that are well known in the art or can be isolated from a biological source. One method for synthesizing oligonucleotide primers on a solid support is disclosed in U.S. Pat. No. 4,458,068 the disclosure of which is herein incorporated by reference into the present application.

The term “nucleic acid” includes double or single stranded DNA or RNA or a double stranded DNA-RNA hybrid and/or analogs and derivatives thereof. In the context of PCR a “template molecule” may represent a fragment or fraction of the nucleic acids added to the reaction. Specifically, a “template molecule” refers to the sequence between and including the two primers. The nucleic acid of specific sequence may be derived from any of a number of sources, including humans, mammals, vertebrates, insects, bacteria, fungi, plants, and viruses. In certain embodiments, the target nucleic acid is a nucleic acid whose presence or absence can be used for certain medical or forensic purposes such as diagnosis, DNA fingerprinting, etc. Any nucleic acid can be amplified using the present invention as long as a sufficient number of bases at both ends of the sequence are known so that oligonucleotide primers can be prepared which will hybridize to different strands of the sequence to be amplified.

The term “PCR” refers to a polymerase chain reaction, which is a thermocyclic, polymerase-mediated, DNA amplification reaction. A PCR, typically includes template molecules, oligonucleotide primers complementary to each strand of the template molecules, a thermostable DNA polymerase, and deoxyribonucleotides, and involves three distinct processes that are multiply repeated to effect the amplification of the original nucleic acid. The three processes (denaturation, hybridization, and primer extension) are often performed at distinct temperatures, and in distinct temporal steps. In many embodiments, however, the hybridization and primer extension processes can be performed concurrently.

The term “deoxyribonucleoside triphosphates” refers to dATP, dCTP, dGTP, and dTTP or analogues.

The term “polymerization agent” as used in the present application refers to any compound or system which can be used to synthesize a primer extension product. Suitable compounds include but are not limited to thermostable polymerases, E. coli DNA polymerase I, Klenow fragment of E. coli DNA polymerase I, T4 DNA polymerase, T7 DNA polymerase, T. litoralis DNA polymerase, and reverse transcriptase.

A “thermostable polymerase” refers to a DNA or RNA polymerase enzyme that can withstand extremely high temperatures, such as those approaching 100° C. Often, thermostable polymerases are derived from organisms that live in extreme temperatures, such as Thermus aquaticus. Examples of thermostable polymerases include, Taq, Tth, Pfu, Vent, deep vent, UlTma, and variations and derivatives thereof

“E. coli polymerase I” refers to the DNA polymerase I holoenzyme of the bacterium Escherichia coli.

The “Klenow fragment” refers to the larger of two proteolytic fragments of DNA polymerase I holoenzyme, which fragment retains polymerase activity but which has lost the 5′-exonuclease activity associated with intact eke.

“T7 DNA polymerase” refers to a DNA polymerase enzyme from the bacteriophage T7.

A “target nucleic acid” refers to a nucleic acid of specific sequence, derived from any of a number of sources, including humans, mammals, vertebrates, insects, bacteria, fungi, plants, and viruses. In certain embodiments, the target nucleic acid is a nucleic acid whose presence or absence can be used for certain medical or forensic purposes such as diagnosis, DNA fingerprinting, etc. The target nucleic acid sequence may be contained within a larger nucleic acid. The target nucleic acid may be of a size so ranging from about 30 to 1000 base pairs or greater. The target nucleic acid may be the original nucleic acid or an amplicon thereof.

A “non-target nucleic acid” refers to a nucleic acid of specific sequence, derived from any of a number of sources, including humans, mammals vertebrates, insects, bacteria, film plants, and viruses that can be primed by the using the same primer or primers as the target nucleic acid. In certain embodiments, the non-target nucleic acid is a nucleic acid whose presence or absence can be used for certain medical or forensic purposes such as diagnosis, DNA, fingerprinting, etc. The non-target nucleic acid may be a sequence that is unconverted or partially converted following the a chemical reaction designed to convert one or more bases in a nucleic acid sequence. The non-target nucleic acid sequence may be contained within a larger nucleic acid. The non-target nucleic acid may be of a size ranging from about 30 to 1000 base pairs of greater. The non-target nucleic acid may be the original nucleic acid or an amplicon thereof.

In order that the present invention mazy be more readily understood, we provide the following non-limiting examples.

### BRIEF DESCRIPTION OF THE DRAWINGS

FIG. 1 shows aligned sequences of the amplified region of the16S ribosomal RNA genes from E. coli, Salmonella and Sulfobacillus thermsulfidooxidans. Bases identical in all three species are shaded black and those identical in just E. coli and Salmonella in grey. The sequences corresponding to the primers are indicated.

FIG. 2 amplification of bacterial rDNAs using different denaturation temperatures. DNA from different bacterial species was amplified using the primers NR-Fli and N-Rli as described in the text. Amplifications were done across a denaturation temperature range of 84.4° C. to 92.8° C. Temperatures of individual reactions were 84.4° C., 85.7° C., 87.2° C., 88.7° C., 90.2° C., 91.6° C. and 92.8° C. Reaction products were analysed on a 1.5% agarose gel and the lowest temperature at which amplification was observed for each species is indicated.

FIG. 3 Amplification of E. coli DNA in the presence of excess S. thermosulfidooxidans rDNA. Mixes of E. coli and S. thermosulfidooxidans rDNA in the ratios indicated in the panels were amplified by PCR using denaturation temperatures of 91.6° C. or 87.2° C. Melting profiles of the amplification products were done using SybrGreen in an Applied Biosystems ABI PRISM 7700 Sequence Detection System. The right hand arrowed peak corresponds to the S. thermosulfidooxidans rDNA amplicon and the left arrowed peak to the E. coli rDNA amplicon. The broad peak to the left, between 70° C. and 80° C. corresponds to primer dimers. In each panel the trace that exhibits a peak for S. thermosulfidooxidins rDNA is from the 91.6° C. amplification and the other trace, lacking this peak, is of the 87.2° C. amplification.

FIG. 4 DNA from mixtures of bacteria as described in the text was amplified using a denaturation temperature of 86.3° C. Radiolabeled reaction products were digested with Taq1 that distinguishes E. coli and Salmonella amplicons Products were analysed by electrophoresis on a 10% polyacylamide, 7M urea gel. Arrows indicate the position of restriction fragments derived from the Salmonella rDNA amplicon and asterisks those from the E. coli amplicon.

FIG. 5 shows the sequence of the promoter region of the GSTP1 gene before and after reaction with sodium bisulphite; and

FIG. 6 is a series of graphs showing the effect of varying denaturation temperature on amplification of unconverted and bisulphite-converted methylated and unmethylated GSTP1 promoter sequences.

### DETAILED DESCRIPTION OF THE INVENTION

### EXAMPLE 1

### Selective Amplification of Specific Bacterial DNAs

To demonstrate that the invention can be applied to any type of DNA sequence we have shown how it can be applied for the differential amplification of ribosomal DNAs from different bacterial species.

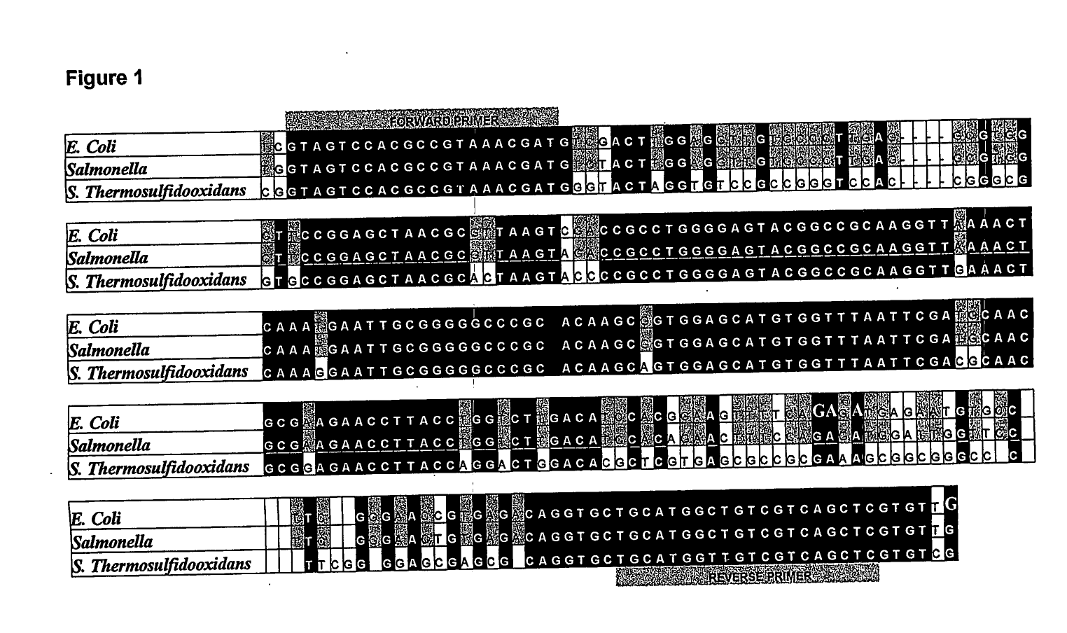

Amplification of 16S ribosomal DNAs is often used in the identification of bacterial species and sequences of a large number of species have been determined. The presence of certain highly conserved regions has allowed the design of primer pairs for the amplification of essentially all bacterial ribosomal DNAs. FIG. 1 shows the sequences of the target region of 16S ribosomal RNAs of three bacterial species. E. coli, Salmonella and Sulfobacillus thermosulfidooxidans and the regions to which the primers bind. Bacterial rDNA from each species was amplified using the forward and reverse primers:

| | |
|---|---|
| NR-F1i | 5′- GTA GTC CII GCI ITA AAC GAT - 3′ |
| NR-R1i | 5′- GAG CTG ICG ACI ICC ATG CA - 3′ |

(I = inosine)

PCR reactions were set up in 25 μl containing

| | |
|---|---|
| 2x PCR master Mix (Promega) | 12.5 μl |
| Forward primer | 0.8 μl |
| Reverse primer | 0.8 μl |
| DNA | 1.0 μl |
| Water | 9.9 μl |

Reactions were run on an Eppendorf Mastercycler intent. After 4 cycles in which a high (95° C.) denaturation temperature was used, subsequent cycles employed a temperature gradient across the block for the denaturation step. The higher temperature in early rounds is to ensure full denaturation of longer genomic DNA fragments prior to the presence of a defined size PCR product. Cycling conditions were as follows:

| | | | |
|---|---|---|---|
| 95° C. | 2 min | | |
| 95° C. | 30 sec | | |
| 58° C. | 30 sec | | |
| | | {close oversize brace} | 4 cycles |
| 72° C. | 1 min | | |
| X ° C. | 30 | | (temperatures as |
| | sec | {close oversize brace} | indicated in figures and text) |
| 58° C. | 30 sec | | 30 cycles |
| 72° C. | 1 min | | |
| 72° C. | 5 min | | |

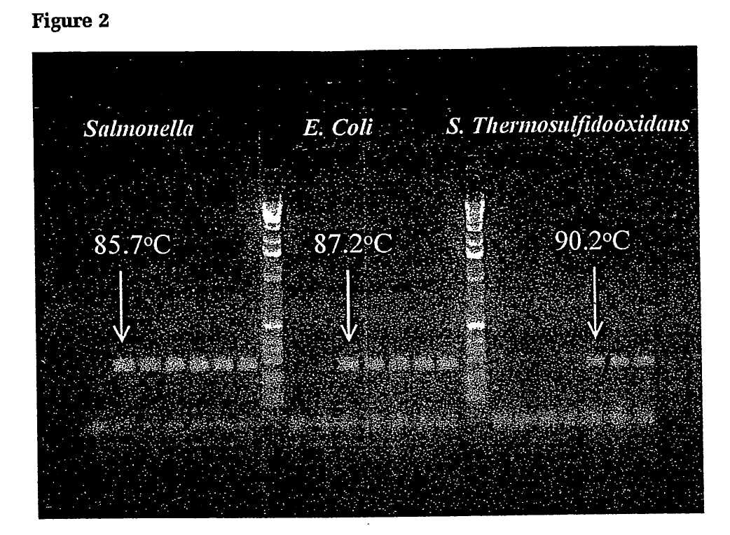

PCR reactions across a range of denaturation temperatures from 84° C. to 93° C. were analysed by agarose gel electrophoresis (FIG. 2). rDNA from S. thermosulfidooxidans is only amplified in reactions where the denaturation temperature is 90.2° C. or greater, E. coli at temperatures above 87.2° C. and Salmonella above 85.7° C. The G+C content of the S. thermosulfidooxidans, E. coli and Salmonella amplicons are 63.2%, 55.4% and 53.9% respectively. The 271 bp E. coli amplicon has only 4 more G/C pairs than Salmonella, yet this provides a sufficient difference in denaturation temperature to allow selective amplification of Salmonella rDNA.

### Amplification of Mixtures of E. coli and S. thermosulfidooxidans DNA

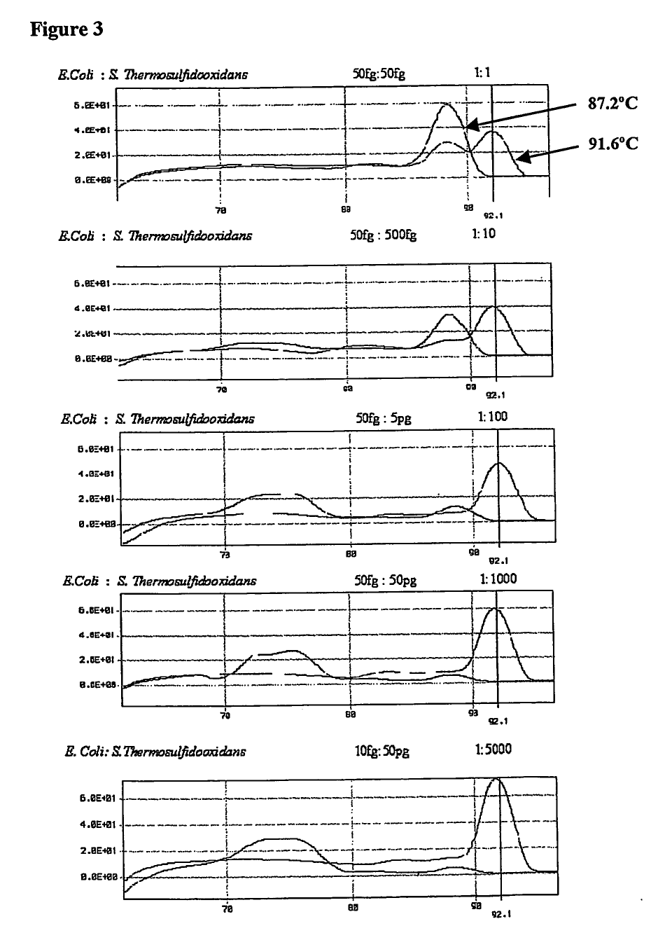

Selective amplification of E. coli rDNA in the presence of a large excess of DNA from S. thermosulfidooxidans is demonstrated in FIG. 3. 50 fg of the E. coli rDNA amplicon was mixed with increasing amounts of the S. thermosulfidooxidans amplicon (50 fg to 50 pg) giving ratios of 1:1 to 1:1000, as well as a 10 fg:50 pg (1:5000). Following amplification for 30 cycles using denaturation temperatures of either 87.2° C. or 91.2° C. When the higher donation temperature is used the relative amounts of amplification product identified from the melting curves approximates the input levels of E. coli and S. thermosulfidooxidans DNA—equivalent levels in the top panel, some E. coli amplicon evident when input in ratio 1:10 and essentially only a peak for S. thermosulfidooxidans with ratios of 1:100 and above. Performing the PCR with a denaturation temperature of 87.2° C. results in a dramatic shift in the profile of amplification products. There is essentially no amplicon produced with a melting profile corresponding that of S. thermosulfidooxidans even when it is present in 5000 fold excess in the input DNA Amplification of E. coli DNA is evident at all input ratios, though the amplification of substantial amounts of primer-dimer (broad peak to the left of melting profile) appears to have limited the final level of amplification of the E. coli product. It is clear that at least a 5000 fold preferential amplification of E. coli rDNA compared to S. thermosulfidooxidans can be obtained by selecting a denaturation temperature for PCR that is below the melting temperature of the S. thermosulfidooxidans rDNA amplicon.

### Detection of Salmonella in the Presence of Excess i E. coli

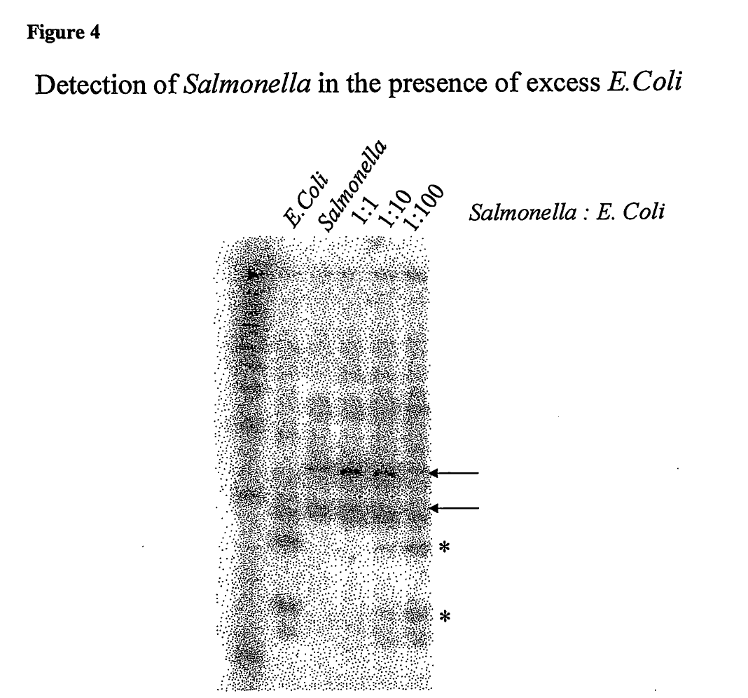

Differential melting temperature PCR was applied to DNA from mixes of different proportions of E. coli and Salmonella bacteria. Mixtures were made of 10⁴ salmonella with 10⁴, 10⁵ and 10⁶ E. coli in 50 μl of 10 mM Tris, pH 8.0, 1 mM EDTA and the mixtures boiled for 10 min. Bacterial debris was removed by centrifugation in a microfuge for 15 min. 4 μl of each supernatant was added to a PCR mix and PCR done as above with a denaturation temperature of 86.3° C. Products were analysed by restriction digestion after incorporation of α-³²P dATP through 4 extra cycles of PCR using a non-selective, 95° C., denaturation temperature. Restriction fragments (FIG. 4) corresponding to the Salmonella amplicon (arrows) predominate at ratios of 1:1 and 1:10, but are in the minority relative to the E. coli amplicon (asterisked bands) when the ratio of Salmonella to E. coli DNA is 1:100. The data indicates an approximately 30 fold preferential amplification of the Salmonella rDNA amplicon. Given the small difference in melting temperature, it should be possible to obtain greater differential amplification by choosing primers to generate a much smaller amplicon with maximal differences between the species.

### EXAMPLE 2

When DNA is treated with sodium bisulphite cytosines (Cs) are converted to uracil (U) while methyl cytosines (meC) remain unreactive. During DNA amplification by PCR, Us are replaced by thymines (Ts); meCs remain as Cs in the amplified DNA. In mammalian DNA most meC is found at CpG sites. At particular sites or regions CpGs may be either methylated or unmethylated. Following bisulphite treatment Cs that are part of CpG sites may be either C or U, while other Cs should be converted to U. Because of incomplete denaturation or secondary structure, reaction of DNA with bisulphite is not always complete and, depending on primers and PCR conditions, unmodified or partially modified DNA may be amplified. This can particularly be the case when using “methylation specific PCR” primers as they are generally designed to amplify molecules containing methylated cytosines (i.e. not converted) adjacent to the priming sites. In amplifying methylated sequences of the GSTP1 gene we found unwanted amplification of un- or incompletely converted DNA in some DNA samples and that this amplification could suppress amplification of true methylated molecules present in the population. In this example we show that the use of a lower denaturation temperature can suppress amplification of unconverted DNA that has a significantly higher melting temperature.

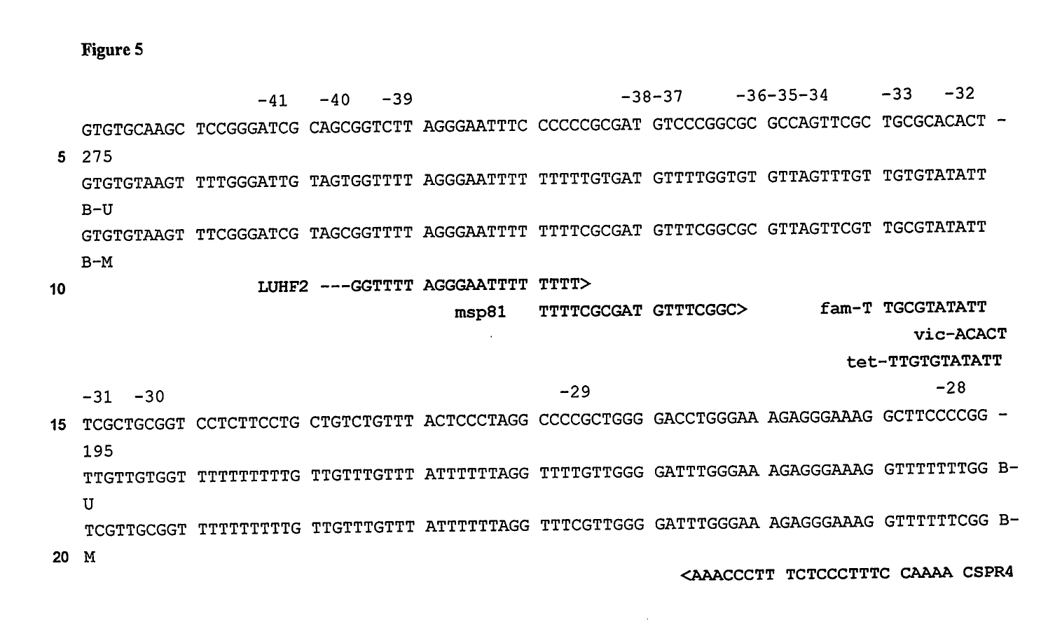

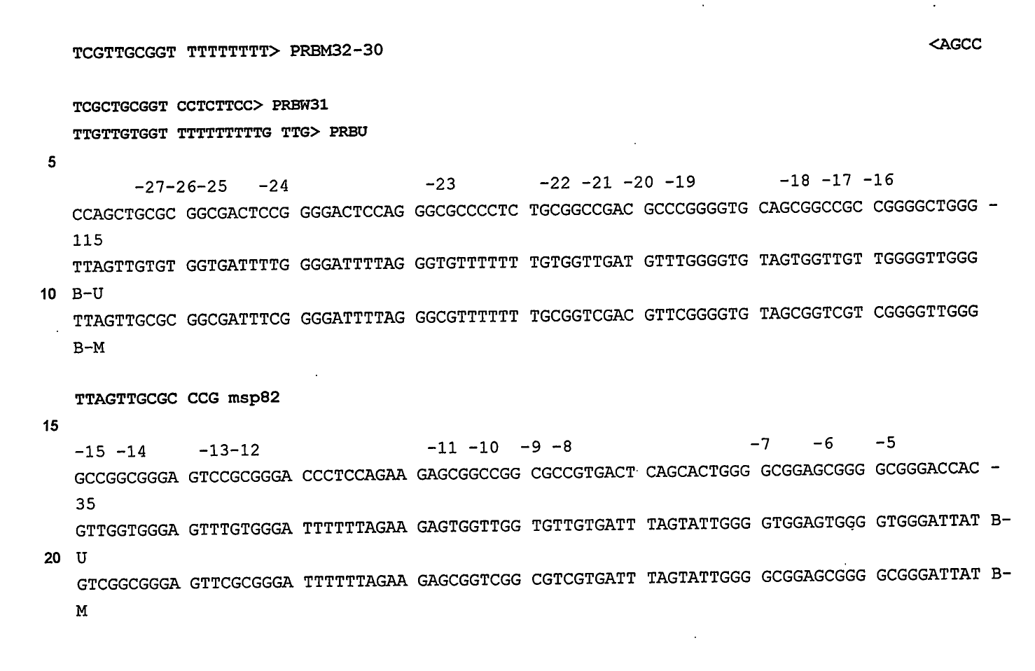

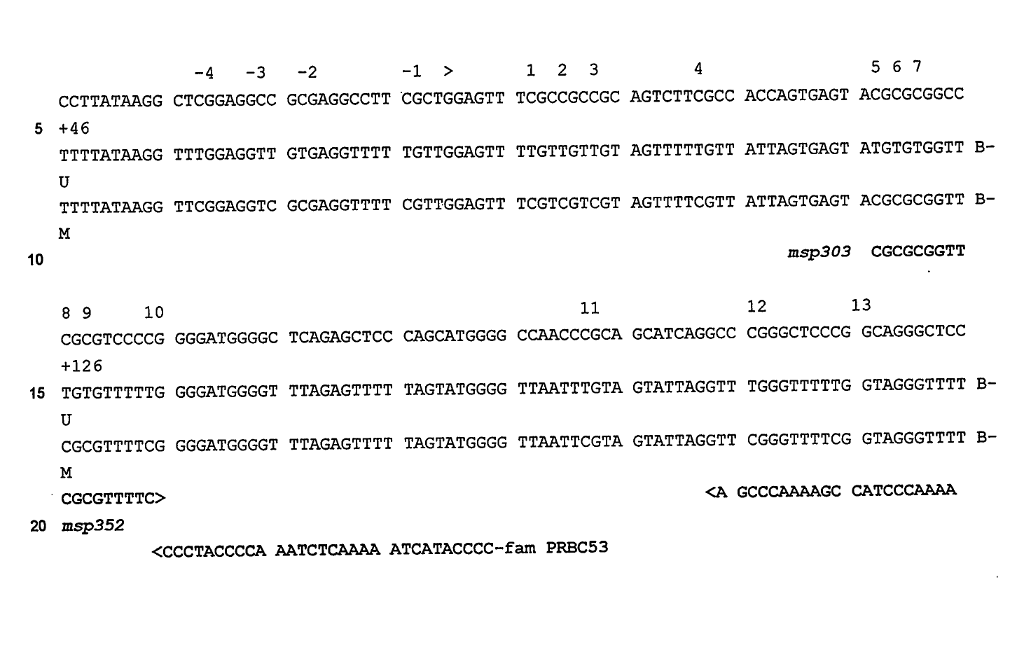

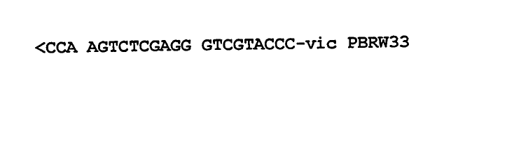

The sequence of promoter region and 3′ to the transcription start site of the GSTP1 gene is shown in FIG. 5; numbering of the sequence and of CpG sites is relative to the transcription start site. The upper line shows the unmodified sequence and the next two lines the sequence after reaction with sodium bisulphite assuming the CpG sites are either unmethylated (B-U) or methylated (B-M) respectively. The positions of primers and Taman probes used in this and subsequent examples are shown.

To demonstrate the principle of the invention, we took a mixture of amplified GSTP1 DNA that contained sequences corresponding to unmethylated DNA, methylated DNA and unconverted DNA This was amplified using the primers and TaqMan probes shown in the table below. Note that primer LUH F2 contains a 5′ “tail” that is designed to suppress amplification of unmethylated DNA (unpublished results), but this is independent of melting temperature effects demonstrated here.

| Primer/ | |
|---|---|
| probe | Sequence |
| LUHF2 | 5′ACACCAAAACATCACAAAAGGTTTTAGGGAATTTTTTTT |
| CSPR4 | 5′ AAAACCTTTCCCTCTTTCCCAAA |
| PRBM32-30 | fam-T TGCGTATATTTCGTTGCGGTTTTTTTTT-TAMRA |
| PRBW31 | vic-ACACTTCGCTGCGGTCCTCTTCC-TAMRA |
| PRBU | tet-TTGTGTATATTTTGTTGTGGTTTTTTTTTTGTTG- |
| | TAMRA |

25 μl reactions contained:
Platinum Taq PCR buffer (Promega)
Platinum Taq (0.25 μ)
Primers LUHF2 (200 nM) and CSPR4 (40 nM)
dCTP, dGTP, dATP and dUTP (200 μM)

### Amplification Conditions: 50° C. 2 min

95° C. 2 min
95° C. 15 sec, 60° C. 1 min 5 cycles
XX ° C. for 15 sec, 60° C. 1 min 40 cycles. (XX−different temperature)

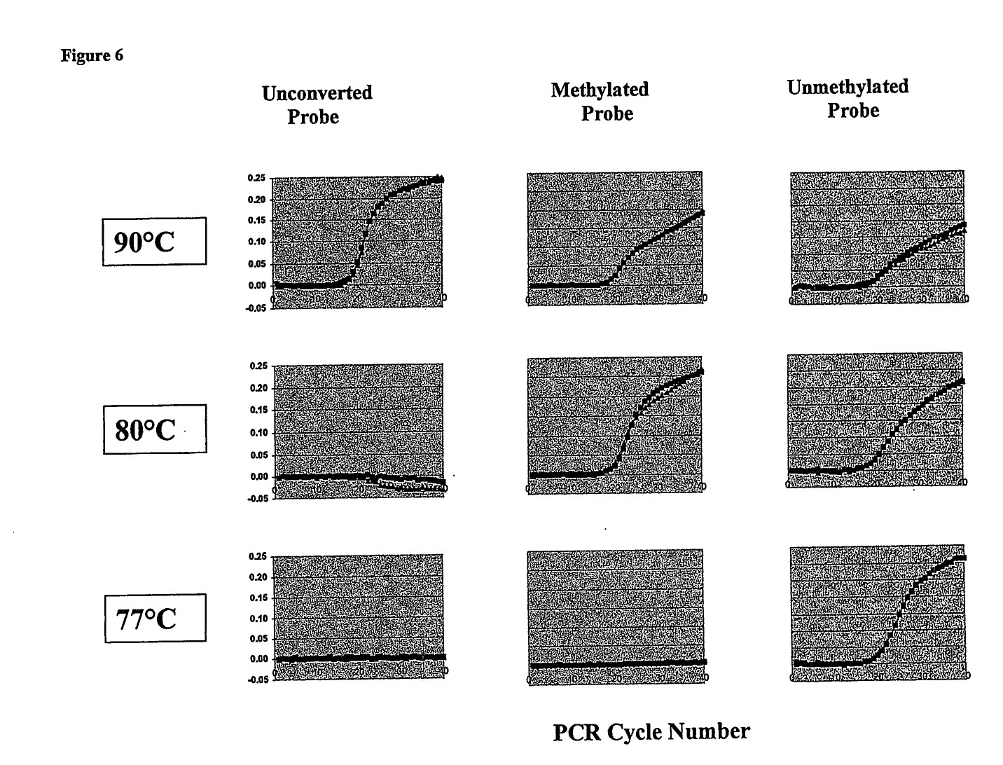

Amplifications were done in an Applied Biosystems 7700 instrument and reaction products followed by release of fluorescent probes. The probes PRB-M, PRB-U and PRB-W respectively detect methylated, unmethylated and unconverted DNA. Amplifications were done using 5 initial cycles with denaturation at 95° C. in order that longer stating DNA molecules were fully denatured before lowering the denaturation temperature for subsequent cycles. The results of amplifications with different denaturation temperatures are shown in FIG. 6.

When PCR is performed using a denaturation temperature of 90° C. amplification of all three templates detected. Reduction of the denaturation temperature to 80° C. prevents amplification of unconverted DNA, while allowing amplification of both methylated and unmethylated DNA products with efficiency equivalent to that seen with 90° C. denaturation temperature. Further reduction of the denaturation temperature to 77° C. prevents amplification of the methylated DNA product without inhibition of amplification of the unmethylated product. The methylated and unmethylated products differ by ten bases in the 141 bp amplicon.

### EXAMPLE 3

The reduced denaturation temperature PCR conditions were applied to a set of patient DNA samples that had shown amplification of unconverted DNA when the normal denaturation temperature of 95° C. was used. Samples of first round PCR product amplified using an outside set of primers, were analysed under conditions equivalent to Example 2 except that primers, msp81 and msp82 were used. Denaturation was at either 95° C. or 80° C. The cycle number at which PCR product reached a threshold level for each sample and probe is shown in the table below.

| | 95° C. Denaturation | | 80° C. Denaturation | |
|---|---|---|---|---|
| | Methylated | Unconverted | Methylated | Unconverted |
| Sample | Probe | Probe | Probe | Probe |
| 83ES | 40 | 40 | 39 | >50 |
| 90ES | 15 | 29 | 15 | >50 |
| 94ES | 13 | 13 | 14 | >50 |
| 101U | >50 | 27 | >50 | >50 |
| 107ES | >50 | 26 | >50 | >50 |

Use of an 80° C. denaturation temperature effectively suppressed amplification of unconverted DNA and it was not detected up to the endpoint of amplification (50 cycles). Where methylated DNA product was detected the efficiency of amplification was essentially identical at both temperatures, with product appearing at equivalent cycle numbers.

### EXAMPLE 4

The effect of amplification of unconverted DNA on the sensitivity of detection of methylated, fully converted DNA was examined at different denaturation temperatures. Plasmid DNA containing cloned GSTP1 sequences derived by PCR from fully bisulphite-converted, methylated DNA were amplified alone or mixed with 1 μl of a PCR reaction that yielded a high level of unconverted DNA sequences. Both the plasmid DNA and the unconverted DNA were derived using primers outside primers msp81 and msp82 used for PCR amplification, The input of plasmid DNA was varied from zero to 10⁶ copies per PCR reaction. Amplifications were done as in Example 3 and the threshold values at which PCR products were detected is shown in the table below.

| | 95° C. Denaturation | | | | 80° C. Denaturation | | | |
|---|---|---|---|---|---|---|---|---|
| | | | Plasmid & | | | | Plasmid & | |
| | | | Unconverted | | | | Unconverted | |
| | Plasmid | | DNA | | Plasmid | | DNA | |
| DNA | Meth | Unc | Meth | Unc | Meth | Unc | Meth | Unc |
| copies | Probe | Probe | Probe | Probe | Probe | Probe | Probe | Probe |
| 10⁶ | 24.0 | >50 | >50 | 11.1 | 25.4 | >50 | 27.2 | >50 |
| 10⁵ | 27.3 | >50 | >50 | 11.5 | 28.6 | >50 | 30.2 | >50 |
| 10⁴ | 30.9 | >50 | >50 | 11.4 | 31.9 | >50 | 33.0 | >50 |
| 10³ | 34.2 | >50 | >50 | 11.2 | 35.6 | >50 | 37.1 | >50 |
| 10² | 38.2 | >50 | >50 | 11.4 | 40.9 | >50 | 40.6 | >50 |
| 10 | >50 | >50 | >50 | 11.4 | >50 | >50 | >50 | >50 |
| 0 | 40.4 | >50 | >50 | 11.1 | >50 | >50 | >50 | >50 |

When plasmid alone was amplified 100 or more copies were readily amplified both under normal (95° C.) and 80° C. denaturation conditions. In the presence of unconverted DNA, amplification of the unconverted sequences (reaching a threshold of detection by cycle 11 to 12) completely suppressed amplification of the methylated, converted DNA when the denaturation temperature was 95° C. However, when denaturation was at 80° C. amplification of the unconverted DNA was completely suppressed allowing amplification of the methylated, converted DNA. Amplification was slightly less efficient than in the absence of the competing unconverted DNA. Thus, use of the lower denaturation temperature can allow the detection of sequences that would otherwise have been masked by amplification of competing related-sequence DNA.

### EXAMPLE 5

To demonstrate that the same principle can be applied to a separate sequence region, sequences within the transcribed region of the GSTP1 gene were amplified using primers msp303 an msp352 (see FIG. 5). Amplifications were done using two clinical samples one of which had previously shown amplification of unconverted DNA across this region and the other that had been shown to contain methylated, converted sequences only. Threshold cycles of detection of PCR products (in duplicate for each condition) are shown in the table below.

| | 95° C. Denaturation | | 80° C. Denaturation | |
|---|---|---|---|---|
| | Conversion | | Conversion | |
| | Probe | Unconverted | Probe | Unconverted |
| DNA | PRBC53 | Probe PRBW53 | PRBC53 | Probe PRBW53 |
| 85ES | 8, 8 | >50, >50 | 9, 8 | >50, >50 |
| 86U | >50, >50 | 19, 22 | >50, >50 | >50, >50 |

For sample 85ES the correct PCR product is detected after 8 or 9 cycles whether the denaturation temperature is 95° C. or 80° C.; thus amplification is not inhibited at the lower temperature. In contrast amplification of unconverted DNA is seen for sample 86U when the denaturation temperature is 95° C. but this amplification is suppressed when the denaturation temperature is lowered to 80° C.

It will be recognised from the above that the invention of the present application has many possible applications. These include, but are not limited to, selective amplification of DNA and RNA, selection and/or identification of species, suppression of spurious or undesired products in amplification reactions such as PCR, assays for the prognosis and diagnosis of diseases or cancers characterized by abnormal undermethylation of DNA.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Moreover any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

Finally, it will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for the selective amplification of at least one target nucleic acid in a sample comprising the at least one target nucleic acid and at least one non-target nucleic acid, the target nucleic acid having a lower melting point than that of the non-target nucleic acid, the method comprising one or more cycle(s) of a nucleic acid denaturation step followed by an amplification step using at least one amplification primer, wherein the denaturation step is carried out at a temperature at or above the melting temperature of the at least one target nucleic acid but below the melting temperature of the at least one non-target nucleic acid so as to substantially suppress amplification of the non-target nucleic acid.

2. A method according to claim 1, wherein the amplification primer is a forward primer.

3. A method according to claim 1, wherein the amplification primer is a reverse primer.

4. A method according to claim 1, wherein the amplification step uses at least one forward and one reverse primer.

5. A method according to claim 1, wherein the amplification step is selected from the group consisting of polymerase chain reaction (PCR), strand displacement reaction (SDA), nucleic acid sequence-based amplification (NASBA), ligation-mediated PCR, and a rolling-circle amplification (RCA).

6. A method according to claim 5, wherein the amplification step is performed using PCR or the like.

7. A method according to claim 6, wherein said amplification step is performed using real time PCR.

8. A method according to claim 1, wherein the denaturation step is carried out at a temperature between the melting temperature of the target nucleic acid and the non-target nucleic acids.

9. A method according to claim 1, wherein the denaturation step is carried out at a temperature below the melting temperature of the non-target nucleic acid but at or sufficiently above the melting temperature of the target nucleic acid as to allow amplification of the target nucleic acid.

10. A method for selectively amplifying different, but related nucleic acid sequences wherein the difference is one or more deletions, additions and/or base changes between at least one target nucleic acid and at least one non-target nucleic acid, the method comprising the method as defined in any one of the preceding claims.

11. A method of species selection and/or identification in a sample comprising a mixture of nucleic acids obtained from two or more target species (target nucleic acid) and one or more non-target species (non-target nucleic acid), the target nucleic acid having a lower melting point than that of the non-target nucleic acid, the method comprising:
subjecting the sample to one or more cycles of a nucleic acid denaturation step followed by an amplification step using at least one amplification primer, wherein the denaturation step is carried out at a temperature at or above the melting temperature of the at least one target nucleic acid but below the melting temperature of the at least one non-target nucleic acid, so as to substantially suppress amplification of the non-target nucleic acid; and
determining the presence of amplified product

12. A method according to claim 11, wherein the species is selected from animal species, bacterial species, fungal species and plants species.

13. A method according to claim 11, when used for the selection of one or more species in a population of species.

14. A method according to claim 13, when used for the selective amplification of isolated nucleic acid that is a mixture of nucleic acid from a minor species and a dominant species, wherein the melting point of the minor species is lower than that of the dominant species.

15. A method according to claim 11, wherein the nucleic acid is DNA

16. A method according to claim 11, wherein the nucleic acid is RNA.

17. A method according to claim 11, wherein the species is a bacterial species.

18. A method according to claim 11, comprising a method according to claim 1.

19. A method for suppressing or eliminating spurious or undesired amplification product(s) during amplification of a target nucleic acid, where the melting temperature of the undesired products is above that of the target nucleic acid, the method comprising one or more cycle(s) of a nucleic acid denaturation step followed by an amplification step using at least one amplification primer, wherein the denaturation step is carried out at a melting temperature at or above the temperature of the target nucleic acid but below that of the undesired products so as to substantially suppress amplification of the spurious or undesired product.

20. A method according to claim 19, wherein the target nucleic acid has been subjected to chemical treatment to produce a converted nucleic acid and wherein the undesired amplification product is unconverted or partially converted nucleic acid.

21. A method according to claim 20, wherein the target nucleic acid has been subjected to treatment with bisulphite and the undesired amplification product is derived from nucleic acid that is partially or incompletely reacted with bisulphite.

22. A method for the selective amplification of at least one target nucleic acid in a sample comprising the at least one target nucleic acid and at least one non-target nucleic acid, the method comprising the steps:
(a) modifying the target nucleic acid and/or non-target nucleic so as the alter the relative melting temperatures of the target nucleic acid and the non-target nucleic, the melting temperature of the target nucleic acid being below that of the non-target nucleic acid;
(b) amplifying the target nucleic acid by performing one or more cycle(s) of nucleic acid denaturation followed by an amplification step wherein the denaturation step is carried out at a temperature at or above the melting temperature of the target nucleic acid of step (a), but below the melting temperature of the at least one non-target nucleic acid of step (a) so as to substantially suppress amplification of the non-target nucleic acid.

23. A method of claim 22, wherein prior to step (a), the melting temperatures of the at least one target nucleic acid and the at least one non-target nucleic acid are substantially the same and the chemical modification produces a difference in the relative melting temperature of the target nucleic and the non-target nucleic acid.

24. A method according to claim 22, wherein prior to step (a) the target nucleic acid has a lower melting temperature than the non-target nucleic acid and the modification in step (a) increases the melting temperature difference between the target nucleic acid and the non-target nucleic acid.

25. A method according to claim 22, wherein the modification is the conversion of at least one base pair.

26. A method according to claim 22, wherein the modifying agent is a bisulphite.

27. A method according to claim 22, wherein the modification modifies unmethylated cytosine to produce a converted nucleic acid.

28. A method according to claim 1, including the further step of isolating the target nucleic acid(s) and optionally subjecting the isolated target nucleic acid(s) sequence analysis.

29. A method according to claim 22, including the further step of isolationg the target nucleic acid(s) and optionally subjecting the isolated target nucleic acid(s) to sequence analysis.

30. An assay for abnormal under-methylation of a nucleic acid, wherein said assay comprises the steps of:
i) subjecting a sample suspected to contain abnormally under-methylated nucleic acid and optionally methylated nucleic acid to bisulphite treatment;
ii) performing a selective amplification of nucleic acids wherein the selective amplification comprises one or more cycle(s) of a nucleic acid denaturation step, wherein the denaturation is carried out at a temperature at or above the melting temperature of the nucleic acids containing abnormally under-methylated nucleic acids but below the melting temperature of nucleic acids containing methylated nucleic acid; and
iii) determining the presence of amplified nucleic acid.

31. A prognostic or diagnostic assay for a disease or cancer in a subject, said disease or condition characterized by abnormal under-methylation of nucleic acids, wherein said assay comprises the steps of:
i) reacting a sample of nucleic acid(s) taken from the subject with bisulphite
ii) performing a selective amplification of nucleic acids from (i) wherein the selective amplification comprises one or more cycle(s) of a denaturation step prior to an amplification step, wherein the denaturation is carried out at a temperature at or above the melting temperature of target nucleic acid containing abnormally under-methylated nucleic acids but below the melting temperature of non-target methylated or substantially methylated nucleic acid(s) so as to substantially suppress amplification of the non-target nucleic acid; and
iii) determining the presence of amplified nucleic acid.

32. A method according to claim 31, wherein the condition or disease is a cancer.

33. A method according to claim 32, wherein the cancer is selected from lung cancers, breast cancer, cervical dysplasia and carcinoma, colorectal cancer, prostate cancer and liver cancer.

34. A method according to any one of claim 31, wherein the amplification step is selected from the group consisting of polymerase chain reaction (PCR), strand displacement reaction (SDA), nucleic acid sequence-based amplification (NASBA), ligation-mediated PCR, and a rolling-circle amplification (RCA).

35. A method according to claim 34, wherein the amplification step is performed using PCR or the like.

36. A method according to claim 34, wherein said amplification step is performed using real time PCR.


SEQUENCE LISTING (11 sequences)

SEQ ID NO: 1 - DNA, 21 bp, Artificial Sequence
  Chemically-Synthesized Oligonucleotide Primer
gtagtccnngcnntaaacgat

SEQ ID NO: 2 - DNA, 20 bp, Artificial
  Chemically-Synthesized Oligonucleotide Primer
gagctgncgacnnccatgca

SEQ ID NO: 3 - DNA, 39 bp, Artificial Sequence
  Chemically-Synthesized Oligonucleotide Primer
acaccaaaacatcacaaaaggttttagggaatttttttt

SEQ ID NO: 4 - DNA, 23 bp, Artificial Sequence
  Chemically-Synthesized Oligonucleotide Primer
aaaacctttccctctttcccaaa

SEQ ID NO: 5 - DNA, 29 bp, Artificial Sequence
  Chemically-Synthesized Oligonucleotide Primer
ttgcgtatatttcgttgcggttttttttt

SEQ ID NO: 6 - DNA, 23 bp, Artificial Sequence
  Chemically-Synthesized Oligonucleotide Primer
acacttcgctgcggtcctcttcc

SEQ ID NO: 7 - DNA, 34 bp, Artificial Sequence
  Chemically-Synthesized Oligonucleotide Primer
ttgtgtatattttgttgtggttttttttttgttg

SEQ ID NO: 8 - DNA, 279 bp, Escherichia coli
tcgtagtccacgccgtaaacgatgtcgacttggaggttgtgcccttgaggcgtggcttccggagctaacgcgttaagtcgaccgcctggggagtacggccgcaaggttaaaactcaaatgaattgcgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttg

SEQ ID NO: 9 - DNA, 279 bp, Salmonella
tggtagtccacgccgtaaacgatgtctacttggaggttgtgcccttgaggcgtggcttccggagctaacgcgttaagtagaccgcctggggagtacggccgcaaggttaaaactcaaatgaattgcgggggcccgcacaagcggtggagcatgtggtttaattcgatgcaacgcgaagaaccttacctggtcttgacatccacagaactttccagagatggattggttccttcgggaactgtgagacaggtgctgcatggctgtcgtcagctcgtgttg

SEQ ID NO: 10 - DNA, 277 bp, Sulfobacillus thermosulfidooxidans
cggtagtccacgccgtaaacgatgggtactaggtgtccgccgggtccaccgggcggtgccggagctaacgcactaagtaccccgcctggggagtacggccgcaaggttgaaactcaaaggaattgcgggggcccgcacaagcagtggagcatgtggtttaattcgacgcaacgcggagaaccttaccaggactggacacgctcgtgagcgccgcgaaagcggcgggcccttcggggagcgagcgcaggtgctgcatggttgtcgtcagctcgtgtcg

SEQ ID NO: 11 - DNA, 1440 bp, Human
gtgtgcaagctccgggatcgcagcggtcttagggaatttccccccgcgatgtcccggcgcgccagttcgctgcgcacactgtgtgtaagttttgggattgtagtggttttagggaatttttttttgtgatgttttggtgtgttagtttgttgtgtatattgtgtgtaagtttcgggatcgtagcggttttagggaattttttttcgcgatgtttcggcgcgttagttcgttgcgtatatttcgctgcggtcctcttcctgctgtctgtttactccctaggccccgctggggacctgggaaagagggaaaggcttccccggttgttgtggttttttttttgttgtttgtttattttttaggttttgttggggatttgggaaagagggaaaggtttttttggtcgttgcggttttttttttgttgtttgtttattttttaggtttcgttggggatttgggaaagagggaaaggttttttcggccagctgcgcggcgactccggggactccagggcgcccctctgcggccgacgcccggggtgcagcggccgccggggctgggttagttgtgtggtgattttggggattttagggtgtttttttgtggttgatgtttggggtgtagtggttgttggggttgggttagttgcgcggcgatttcggggattttagggcgtttttttgcggtcgacgttcggggtgtagcggtcgtcggggttggggccggcgggagtccgcgggaccctccagaagagcggccggcgccgtgactcagcactggggcggagcggggcgggaccacgttggtgggagtttgtgggattttttagaagagtggttggtgttgtgatttagtattggggtggagtggggtgggattatgtcggcgggagttcgcgggattttttagaagagcggtcggcgtcgtgatttagtattggggcggagcggggcgggattatccttataaggctcggaggccgcgaggccttcgctggagtttcgccgccgcagtcttcgccaccagtgagtacgcgcggccttttataaggtttggaggttgtgaggtttttgttggagttttgttgttgtagtttttgttattagtgagtatgtgtggttttttataaggttcggaggtcgcgaggttttcgttggagtttcgtcgtcgtagttttcgttattagtgagtacgcgcggttcgcgtccccggggatggggctcagagctcccagcatggggccaacccgcagcatcaggcccgggctcccggcagggctcctgtgtttttggggatggggtttagagtttttagtatggggttaatttgtagtattaggtttgggtttttggtagggttttcgcgttttcggggatggggtttagagtttttagtatggggttaattcgtagtattaggttcgggttttcggtagggtttt